(19) United States
(12) Patent Application Publication
(10) Pub. No.: US 2001/0055579 A1
(43) Pub. Date: Dec. 27, 2001

(54) Hair treatment compositions

(75) Inventors: Andrew Richard Avery, Bebington (GB); Andrew Anthony Barnes, Bebington (GB); Andrew Malcolm Murray, Bebington (GB); Malika Punyagupta, Bangkok (TH)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 09/861,487

(22) Filed: May 18, 2001

(30) Foreign Application Priority Data
May 18, 2000 (GB) 0012064.2

(51) Int. Cl. A61K 7/06; A61K 7/11

(52) U.S. Cl. 424/70.11; 424/70.13

(57) ABSTRACT

Hair treatment compositions comprising a surfactant, PTFE particles, and a cationic polymer. The invention also provides for use of a cationic polymer as a deposition aid for PTFE particles dispersed in an aqueous hair treatment composition.

### FIELD OF THE INVENTION

The invention relates to hair treatment compositions for increased deposition of PTFE particles dispersed in the composition onto the hair, which compositions comprise a cationic polymer.

### BACKGROUND AND PRIOR ART

PTFE particles have previously been described in personal care compositions, as follows:

U.S. Pat. No. 3,568,685 describes a composition for straightening hair, consisting of a water-repellent agent, a hardening and adhesive agent, an emollient and a slipping agent which may be any one of a number of fluoro resins, such as vinylidene fluoride resin or PTFE.

U.S. Pat. No. 3,911,106 describes a method of conditioning hair and scalp by rubbing in PTFE of specified molecular weight. The PTFE may be used by itself, in aqueous composition or diluted with a suitable diluent such as fatty alcohol or mineral oil.

PTFE microparticles have been described in U.S. Pat. No. 4,047,537 describes the use of a colloidal aqueous dispersion of PTFE particles as a hair control agent for promoting hair body, fullness and set retention. The PTFE particles may be used directly on the hair or formulated with an oil-free or lower primary alcohol-free liquid which is preferably a water soluble shampoo.

A problem is that to derive any benefit from PTFE in a rinse-off composition, the composition must leave the PTFE on the target surface (i.e. skin or hair) after the product is washed and rinsed off the surface. PTFE is well known for its “non-stick” properties. This means that a considerable amount of the PTFE will be rinsed away with the composition, and there is scope for substantially improving the deposition efficiency. This would provide better conditioning and the option of reducing the level of PTFE in the composition, with consequent coat saving.

The present inventors have found that the efficiency of deposition of PTFE particles from a shampoo can be significantly enhanced by the inclusion in the composition of a cationic polymer. This is surprising in view of the “non-stick” properties of PTFE particles.

Polymers with a cationic charge have been proposed to enhance the amount of certain benefit agents deposited from shampoo. For example cationic guar gum has been described for the enhancement of the deposition of antidandruff particles in U.S. Pat. No. 5,037,818 and for the enhanced deposition of insoluble non-volatile silicone in U.S. Pat. No. 5,086,857. Cationic polymers have been proposed to enhance the deposition of sunscreen materials from a shampoo composition. In EP 386 898 a cationic polygalactomannan gum derivative is used. The use of cationic polymers in shower gels to enhance deposition of silicone oil in also known from EP-A-457 689 (L'Oreal).

None of the above documents disclose or suggest the utility of cationic polymers an a deposition aid for PTFE particles in aqueous hair treatment compositions such as shampoos.

### SUMMARY OF THE INVENTION

The present invention provides, in a first aspect, an aqueous hair treatment composition comprising:

(i) at least one surfactant;

(ii) PTFE particles, and

(iii) a cationic polymer

In a second aspect, the invention provides the use of a cationic polymer as a deposition aid for PTFE particles dispersed in an aqueous hair treatment composition.

### DETAILED DESCRIPTION AND PREFERRED EMBODIMENTS

### PTFE Particles

By “PTFE particles” is meant PTFE containing particles. These may suitably range in size from 0.01 up to 10 microns, preferably from 0.05 up to 10 microns.

Typically the PTFE particles will be dispersed in the composition to form a separate, discontinuous phase from the aqueous, continuous phase of the composition.

Preferably the PTFE particles are present in the composition of the invention in the form of a colloidal dispersion thereof. Typically the primary particle size of the PTFE particles in such a colloidal dispersion will range from 0.05 up to 0.5 microns, with an average diameter of preferably about 0.2 microns.

The particles may be composed entirely of PTFE polymer, or may consist of a composite of PTFE polymer and one or more further polymers such as polyethylene (PE).

By “PTFE polymer” is meant a polymer consisting of:

(a) 95 to 100%, preferably substantially 100% of units derived from tetrafluoroethylene, and

(b) optionally, up to 5%, preferably not more than 2%, of units derived from a copolymerizable monomer, e.g. hexafluoropropylene, perfluorinated vinyl ether, hexafluoroisobutylene, vinylidene fluoride, or an olefin.

A preferred source of PTFE particles for inclusion in compositions of the invention is a pre-formed colloidal dispersion of PTFE particles.

Such dispersions are commercially available, for example those sold by Ausimont under the trade name ALGOFLON, such as ALGOFLON D60G, and aqueous dispersions of PTFE sold by Du Pont such as Teflon® 30_N.

Also suitable is the series of materials sold by Micropowders, Inc. under the tradename MICROSILK, such as MICROSILK 419.

The PTFE particles are typically present in compositions of the invention at a level of from 0.05% to 10%, preferably from 0.1% to 4%, more preferably from about 1% to 2%, by total weight of PTFE particles based on total weight of the composition.

### Cationic Polymer

Compositions according to the present invention comprise a cationic polymer to enhance deposition of the PTFE particles onto the skin or hair.

The cationic polymer may be a homopolymer or be formed from two or more types of monomers. The molecular weight of the polymer will generally be between 5 000 and 10 000 000, typically at least 10 000 and preferably in the range 100 000 to about 2 000 000. The polymers will have cationic nitrogen containing groups such as quaternary ammonium or protonated amino groups, or a mixture thereof.

The cationic charge density of the cationic polymer is suitably at least 0.1 meq/g, preferably above 0.8 or higher. The cationic charge density should typically not exceed 3 meq/g. It in preferably less than 2 meq/g. The charge density can be measured using the Kjeldahl method and should be within the above limits at the desired pH of use, which will in general be from about 3 to 9 and preferably between 4 and 8.

The cationic nitrogen-containing group will generally be present as a substituent on a fraction of the total monomer units of the cationic polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give a polymer having a cationic charge density in the required range.

Suitable cationic polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition. In general secondary and tertiary amines, especially tertiary, are preferred.

Amine substituted vinyl monomers and amines can be polymerized in the amine form and then converted to ammonium by quaternization.

The cationic polymers can comprise mixtures of monomer units derived from amine- and/or quaternary ammonium-substituted monomer and/or compatible spacer monomers.

Suitable cationic polymers include, for example:

copolymers of 1-vinyl-2-pyrrolidine and 1-vinyl-3-methyl-imidazolium salt (e.g. chloride salt), referred to in the industry by the Cosmetic, Toiletry, and Fragrance Association, (CTFA) as Polyquaternium-16. This material is commercially available from BASF Wyandotte Corp. (Parsippany, N.J., USA) under the LUVIQUAT tradename (e.g. LUVIQUAT PC 370);

copolymers of 1-vinyl-2-pyrrolidine and dimethylaminoethyl methacrylate, referred to in the industry (CTFA) as Polyquaternium-11. This material is available commercially from Gaf Corporation (Wayne, N.J., USA) under the GAFQUAT tradename (e.g., GAFQUAT 755N);

cationic diallyl quaternary ammonium-containing polymers including, for example, dimethyldiallyammonium chloride homopolymer and copolymers of acrylamide and dimethyldiallylammonium chloride, referred to in the industry (CTFA) as Polyquaternium 6 and Polyquaternium 7, respectively;

mineral acid salts of amino-alkyl esters of homo-and copolymers of unsaturated carboxylic acids having from 3 to 5 carbon atoms, (as described in U.S. Pat. No. 4,009,256),

cationic polyacrylamides (as described in WO95/22311).

Other cationic polymers that can be used include cationic polysaccharide polymers, such as cationic cellulose derivatives, cationic starch derivatives, and cationic guar gum derivatives.

Cationic polysaccharide polymers suitable for use in compositions of the invention include those of the formula:

A—O—[R—N⁺(R¹)(R²)(R³)X⁻],

wherein: A is an anhydroglucose residual group, such as a starch or cellulose anhydroglucose residual. R is an alkylene, oxyalkylene, polyoxyalkylene, or hydroxyalkylene group, or combination thereof. R¹, R² and R³ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R¹, R² and R³) is preferably about 20 or less, and X is an anionic counterion.

Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e.g. as described in U.S. Pat. No. 3,962,418), and copolymers of etherified cellulose and starch (e.g. as described in U.S. Pat. No. 3,958,581).

A particularly suitable type of cationic polysaccharide polymer that can be used is a cationic guar gum derivative, such as guar hydroxypropyltrimonium chloride (Commercially available from Rhone-Poulenc in their JAGUAR trademark series).

Examples are JAGUAR C13S, which has a low degree of substitution of the cationic groups and high viscosity. JAGUAR C15, having a moderate degree of substitution and a low viscosity, JAGUAR C17 (high degree of substitution, high viscosity), JAGUAR C16, which in a hydroxypropylated cationic guar derivative containing a low level of substituent groups as well as cationic quaternary ammonium groups, and JAGUAR 162 which is a high transparency, medium viscosity guar having a low degree of substitution.

Preferably the cationic polymer is selected from cationic cellulose and cationic guar derivatives. Particularly preferred cationic polymers are JAGUAR C13S, JAGUAR C15, JAGUAR C17 and JAGUAR C16 and JAGUAR C162.

The cationic polymer will generally be present in compositions of the invention at levels of from 0.01 to 5%, preferably from about 0.05 to 1%, more preferably from about 0.08% to about 0.5% by weight.

### Surfactant

Hair treatment compositions according to the invention comprise at least one surfactant.

Shampoo Compositions

A particularly preferred form of composition in accordance with the invention is a shampoo composition.

Anionic Cleansing Surfactant

Shampoo compositions according to the invention will typically comprise one or more anionic cleansing surfactants which are cosmetically acceptable and suitable for topical application to the hair.

Examples of suitable anionic cleansing surfactants are the alkyl sulphates, alkyl ether Sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic cleansing surfactants for use in shampoo compositions of the invention include sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauryl isethionate and sodium N-lauryl sarcosinate. The most preferred anionic surfactants are sodium lauryl sulphate, sodium lauryl ether sulphate(n)EO, (where n ranges from 1 to 3), ammonium lauryl sulphate and ammonium lauryl ether sulphate(n)EO, (where n ranges from 1 to 3).

Mixtures of any of the foregoing anionic cleansing surfactants may also be suitable.

The total amount of anionic cleansing surfactant in shampoo compositions of the invention is generally from 5 to 30%, preferably from 6 to 20%, more preferably from 8% to 16% by weight based on total weight of the shampoo composition.

Co-surfactant

The shampoo composition can optionally include co-surfactants, to help impart aesthetic, physical or cleansing properties to the composition.

A preferred example is an amphoteric or zwitterionic surfactant, which can be included in an amount ranging from 0 to about 8%, preferably from 1 to 4% by weight based on total weight of the shampoo composition.

Examples of amphoteric and zwitterionic surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

Another preferred example is a nonionic surfactant, which can be included in an amount ranging from 0% to about 8% preferably from 2 to 5% by weight based on total weight of the shampoo composition.

For example, representative nonionic surfactants that can be included in shampoo compositions of the invention include condensation products of aliphatic (C₈-C₁₈) primary or secondary linear or branched chain alcohols or phenols with alkylene oxides, usually ethylene oxide and generally having from 6 to 30 ethylene oxide group.

Other representative nonionic surfactants include mono- or di-alkyl alkanolamides. Examples include coco mono- or diethanolamide and coco mono-isopropanolamide.

Further nonionic surfactants which can be included in shampoo compositions of the invention are the alkyl polyglycosides (APGs). Typically, the APG in one which comprises an alkyl group connected (optionally via a bridging group) to a block of one or more glycosyl groups. Preferred APGs are defined by the following formula:

RO—(G)ₙ

wherein R is a branched or straight chain alkyl group which may be saturated or unsaturated and G is a saccharide group.

R may represent a mean alkyl chain length of from about C₅ to about C₂₀. Preferably R represents a mean alkyl chain length of from about C₈ to about C₁₂. Most preferably the value of R lies between about 9.5 and about 10.5. G may be selected from C₅ or C₆ monosaccharide residues, and is preferably a glucoside. G may be selected from the group comprising glucose, xylose, lactose, fructose, mannose and derivatives thereof. Preferably G is glucose.

The degree of polymerisation, n, may have a value of from about 1 to about 10 or more. Preferably, the value of n lies in the range of from about 1.1 to about 2. Most preferably the value of n lies in the range of from about 1.3 to about 1.5.

Suitable alkyl polyglycosides for use in the invention are commercially available and include for example those materials identified as: Oramix NS10 ex Seppic; Plantaren 1200 and Plantaren 2000 ex Henkel.

Other sugar-derived nonionic surfactants which can be included in shampoo compositions of the invention include the C₁₀-C₁₈ N-alkyl (C₁-C₆) polyhydroxy fatty acid amides, such as the C₁₂-C₁₈ N-methyl glucamides, as described for example in WO 92 06154 and U.S. Pat. No. 5,194,639, and the N-alkoxy polyhydroxy fatty acid amides, such as C₁₀-C₁₈ N-(3-methoxypropyl) glucamide.

### Optional Components

As optional components for inclusion in compositions according to the invention may be mentioned the following conventional adjunct materials known for use in cosmetic compositions: suspending agents, thickeners, pearlescing agents, opacifiers, malts, perfumes, buffering agents, colouring agents, emollients, moisturisers, foam stabilisers, sunscreen materials, antimicrobial agents, preservatives, antioxidants, natural oils and extracts, propellants.

The invention will now be further illustrated by the following, non-limiting Examples.

### EXAMPLE

Two formulations were prepared having ingredients as shown in the following Table. Formulation 1 in a comparative example and Formulation 2 is an example according to the invention.

| Ingredient | Formulation 1 | Formulation 2 |
|---|---|---|
| Chemical Name | a.i. % | a.i. % |
| SLES 2EO | 14 | 14 |
| Cocoamidopropylbetaine | 2 | 2 |
| Guar | 0 | 0.1 |
| hydroxypropyltrimonium | | |
| chloride | | |
| Polytetrafluoroethylene | 2.4 | 2.4 |
| (PTFE) | | |
| Water | to 100 | to 100 |

Comparative testing of formulations 1 and 2 showed that the presence of cationic polymer as in formulation 2 significantly increased PTFE deposition onto hair, relative to that observed for formulation 1. Deposition was assessed by image analysis of particles detected by SEM. Furthermore, the friction coefficient of hair treated with formulation 2 was found to be significantly reduced compared with hair treated with formulation 1. Friction coefficients were measured using a friction rig developed for measurement on switches of hair fibres sliding against skin. Multiple measurements of frictional force were made on multiple switches treated with a particular formulation at each applied load. These data were used to produce representative coefficients of friction for each treatment.

What is claimed is:

1. An aqueous hair treatment composition comprising:
(i) at least one surfactant;
(ii) PTFE particles, and
(iii) a cationic polymer.

2. A composition according to claim 1, in which said cationic polymer is polymer is selected from the group consisting of cationic cellulose and cationic guar derivatives.

3. A composition according to claim 1 or 2, which is in the form of a shampoo composition comprising one or more anionic cleansing surfactants.

4. A composition according to claim 1, in which said PTFE particles are present in the form of a colloidal dispersion thereof.

5. A composition according to claim 4, in which the primary particle size of said PTFE microparticles ranges from 0.05 up to 0.5 microns.

6. Use of a cationic polymer as a deposition aid for PTFE particles dispersed in an aqueous hair treatment composition.
